(19) United States
(12) Patent Application Publication
(10) Pub. No.: US 2011/0178165 A1
(43) Pub. Date: Jul. 21, 2011

(54) TETRAHYDROFURANYL SULFONAMIDES AND PHARMACEUTICAL COMPOSITIONS THEREOF

(75) Inventors: Kimberly Gail Estep, Groton, CT (US); Christopher John O'Donnell, Mystic, CT (US); Longfei Xie, Groton, CT (US)

(73) Assignee: Pfizer Inc.

(21) Appl. No.: 13/122,047

(22) PCT Filed: Sep. 25, 2009
(86) PCT No.: PCT/IB09/54201

§ 371 Date: Mar. 31, 2011

Related U.S. Application Data
(60) Provisional application No. 61/103,656, filed on Oct. 8, 2008.

(51) Int. Cl. A61K 31/381; C07D 307/02; A61K 31/34; C07D 409/10; A61P 25/00; A61P 25/18; A61P 9/00; A61P 25/28; A61P 25/16; A61P 21/00; A61P 25/08; A61P 25/06; A61P 1/00; A61P 25/30; A61P 25/22; A61P 27/16; A61P 27/02; A61P 25/20

(52) U.S. Cl. 514/444; 549/475; 514/473; 549/60

(57) ABSTRACT

The invention is directed to a class of compounds, including the pharmaceutically acceptable salts of the compounds, having the structure of formula (I): as defined in the specification. The invention is also directed to compositions containing the compounds of formula (I). They are useful in the treatment of CNS disorders.

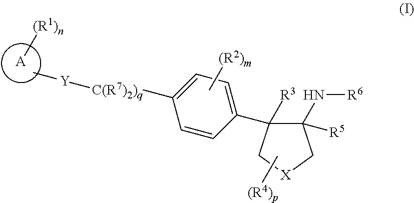

### FIELD OF THE INVENTION

The present invention relates to a novel class of compounds having the structure of formula I as defined herein and pharmaceutical compositions comprising a compound of formula I. The present invention also comprises methods of treating a subject by administering a therapeutically effective amount of a compound of formula I to the subject. These compounds are useful for the conditions disclosed herein. The present invention further comprises methods for making the compounds of formula I and corresponding intermediates.

### BACKGROUND OF THE INVENTION

The present invention provides compounds of Formula I, pharmaceutical compositions thereof, and methods of using the same, processes for preparing the same, and intermediates thereof.

The primary excitatory neurotransmitter in the mammalian central nervous system (CNS) is the amino acid glutamate whose signal transduction is mediated by either ionotropic or metabotropic glutamate receptors (GluR). Ionotropic glutamate receptors (iGluR) are comprised of three subtypes differentiated by their unique responses to the three selective iGluR agonists α-amino-3-hydroxy-5-methyl isoxazole-4-propionic acid (AMPA), N-methyl-D-aspartate (NMDA) and kainate (Parsons, C. G., Danysz, W. and Lodge, D. (2002), in: Ionotropic Glutamate Receptors as Therapeutic Targets (Danysz, W., Lodge, D. and Parsons, C. G. eds), pp 1-30, F.P. Graham Publishing Co., Tennessee). AMPA receptors, proteinaceous homo- or heterotetramers comprised of any combination of four ca. 900 amino acid monomer subunits each encoded from a distinct gene (Glu_{A1-A4}) with each subunit protein existing as one of two splice variants deemed “flip” and “flop”, mediate the vast majority of excitatory synaptic transmissions in the mammalian brain and have long been proposed to be an integral component of the neural circuitry that mediates cognitive processes (Bleakman, D. and Lodge, D. (1998) Neuropharmacology of AMPA and Kainate Receptors. Neuropharmacology 37:1187-1204). The combination of various heterotetrameric possibilities, two splice forms for each of the four iGluR monomers and receptor subunit RNA editing with the heterogeneous distribution of AMPA receptors throughout the brain highlight the myriad of potential AMPA receptor responses within this organ (Black, M. D. (2005) Therapeutic Potential of Positive AMPA Modulators and Their Relationship to AMPA Receptor Subunits. A Review of Preclinical Data. Psychopharmacology 179:154-163). AMPA modulators have now become an active target for drug discovery (see Rogers, B. and Schmidt, C., (2006) Novel Approaches for the Treatment of Schizophrenia, Annual Reports in Medicinal Chemistry 3-21).

### SUMMARY OF THE INVENTION

The present invention is directed to a class of compounds, including the pharmaceutically acceptable salts of the compounds, having the structure of formula:

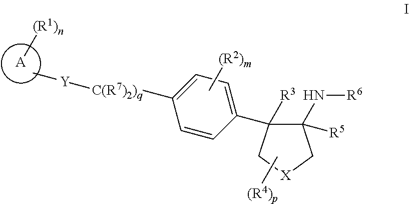

wherein each R¹ and each R² and each R⁷ is independently selected from the group consisting of hydrogen, halogen, hydroxyl, (C₁-C₆)alkoxy, cyano, nitro, amino, (C₁-C₆)alkylamino, di(C₁-C₆)alkylamino, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, (C₆-C₁₀)aryl, (C₁-C₆)heteroaryl, (C₁-C₆)heterocycloalkyl, (C₃-C₁₀)cycloalkyl, or (C₁-C₆)alkyl-S(O)₂—NH—, wherein said (C₁-C₆)alkoxy, (C₁-C₆)alkylamino, di(C₁-C₆)alkylamino, —(C═O)NH((C₁-C₆)alkyl), —(C═O)N—((C₁-C₆)alkyl)₂, —(C═O)O—(C₁-C₆)alkyl, (C₁-C₆)alkyl, (C₆-C₁₀)aryl, (C₁-C₆)heteroaryl, (C₁-C₆)heterocycloalkyl, (C₃-C₁₀)cycloalkyl or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl, (C₃-C₁₀)cycloalkyl, (C₃-C₁₀)cycloalkenyl, (C₁-C₆)heterocycloalkyl, (C₆-C₁₀)aryl, (C₁-C₆)heteroaryl, —(C═O)R⁹, —(C═O)OR⁹, —O(C═O)OR⁹, —(C═O)—N(R⁹)₂, —SO₂—N(R⁹)₂, —N(R⁹)₂, —NR⁹—(C═O)R⁹, and —N(R⁹)—S(O)₂R⁹ wherein each of the R⁸ (C₁-C₆)alkyl, (C₁-C₉)heterocycloalkyl, (C₃-C₁₀)cycloalkyl, (C₆-C₁₀)aryl or (C₁-C₉)heteroaryl is optionally independently substituted with one or more substituents independently selected from the group consisting of halogen, cyano, —R⁹, —OR⁹, —N(R⁹)₂, —S(O)ₜR⁹, —S(O)₂N(R⁹)₂, —N(R⁹)—SO₂R⁹, —O(C═O)R⁹, —(C═O)—OR⁹, —(C═O)—N(R⁹)₂, —N(R⁹)—(C═O)—R⁹, —N(R⁹)—(C═O)—N—(R⁹)₂, and —(C═O)R⁹;

t is 0, 1 or 2;

or when R¹ is (C₆-C₁₀)aryl or (C₁-C₆)heteroaryl, two R⁸ substituents bonded to adjacent carbon atoms of R¹, together with the adjacent carbon atoms, may be taken together to form a (C₁-C₆)heterocyclic or (C₃-C₁₀)carbocyclic ring which is optionally substituted with one or more R¹⁹, wherein each R¹⁰ is independently selected from the group consisting of hydrogen, —CN, halogen, —(C═O)R⁹, —(C═O)—N(R⁹)₂, —N(R⁹)₂, —OR⁹ or —R⁹;

or, two R¹ substituents bonded to adjacent carbon atoms of ring “A,” may be taken together with the adjacent carbon atoms, form a (C₁-C₆)heterocyclic or (C₃-C₁₀)carbocyclic ring which is optionally substituted with one or more R¹⁰;

m is zero, one, two or three;

n is zero, one, two or three;

p is zero, one, two or three;

q is zero, one, two or three;

R³ is hydroxyl;

each R⁴ is independently selected from the group consisting of hydrogen, hydroxyl, (C₁-C₆)alkoxy, cyano, nitro, —(C═O)NH₂, —(C═O)NH((C₁-C₆)alkyl), —(C═O)N((C₁-C₆)alkyl)₂, —O(C═O)(C₁-C₆)alkyl, —(C═O)—O—(C₁-C₆)alkyl, (C₁-C₆)alkyl, (C₁-C₆)alkyl-S(O)₂—NH— or two R⁴ groups on the same carbon atom may be taken together to form an oxo (═O) radical; wherein said (C₁-C₆)alkoxy, —(C═O)NH(alkyl), —(C═O)N-(alkyl)₂, —(C═O)O—(C₁-C₆)alkyl, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl, —(C═O)R⁹, —(C═O)OR⁹, —O(C═O)OR⁹, —(C═O)—N(R⁹)₂, —SO₂—N(R⁹)₂, —N(R⁹)₂, —NR⁹—(C═O)R⁹, and —N(R⁹)—S(O)₂R⁹ wherein each of the R⁸ (C₁-C₆)alkyl is optionally independently substituted with one or more substituents independently selected from the group consisting of halogen, cyano, —R⁹, —OR⁹, —N(R⁹)₂, —S(O)_{q}R⁹, —S(O)₂N(R⁹)₂, —N(R⁹)—SO₂R⁹, —O(C═O)R⁹, —(C═O)—OR⁹, —(C═O)—N(R⁹)₂, —N(R⁹)—(C═O)—R⁹, —N(R⁹)—(C═O)—N—(R⁹)₂, and —(C═O)R⁹;

R⁵ is hydrogen,

R⁶ is (C₁-C₆)alkyl-(C═O)—, [(C₁-C₆)alkyl]₂N—(C═O)—, (C₁-C₆)alkyl-SO₂—, (C₃-C₁₀)cycloalkyl-SO₂—, or [(C₁-C₆)alkyl]₂N—SO₂—; wherein said (C₁-C₆)alkyl moieties of said [(C₁-C₆)alkyl]₂N—(C═O)- and [(C₁-C₆)alkyl]₂N—SO₂— may optionally be taken together with the nitrogen atom to which they are attached to form a three to six membered heterocyclic ring;

R⁸ is independently selected from the group consisting of halogen, —CN, —OR⁹, (C₁-C₆)alkyl, (C₂-C₆)alkenyl, (C₃-C₁₀)cycloalkyl, (C₃-C₁₀)cycloalkenyl, (C₁-C₆)heterocycloalkyl, (C₆-C₁₀)aryl, (C₁-C₉)heteroaryl, —(C═O)R⁹, —(C═O)OR⁹, —O(C═O)OR⁹, —(C═O)N(R⁹)₂, —SO₂NR⁹, —N(R⁹)₂, —N(R⁹)—(C═O)R⁹, and —N(R⁹)₂—SO₂R⁹ wherein each of the R⁸ (C₁-C₆)alkyl, (C₁-C₆)heterocycloalkyl, (C₃-C₁₀)cycloalkyl, (C₆-C₁₀)aryl or (C₁-C₉)heteroaryl is optionally independently substituted with one or more substituents independently selected from the group consisting of halogen, cyano, —R⁹, —OR⁹, —N(R⁹)₂, —S(O)_{q}R⁹, —SO₂N(R⁹)₂, —NR⁹SO₂R⁹, —O(C═O)R⁹, —(C═O)OR⁹, —(C═O)N(R⁹)₂, —NR⁹(C═O)R⁹, —(NR⁹)—(C═O)N(R⁹)₂, and —(C═O)R⁹;

R⁹ is independently selected from the group consisting of hydrogen, (C₁-C₆)alkyl, (C₂-C₆)alkenyl, (C₂-C₆)alkynyl, (C₆-C₁₀)cycloalkyl, (C₆-C₁₀)aryl, (C₁-C₉)heterocycloalkyl and (C₁-C₉)heteroaryl; wherein each R⁹ (C₁-C₆)alkyl, (C₂-C₆)alkenyl, (C₂-C₆)alkynyl, (C₃-C₁₀)cycloalkyl, (C₆-C₁₀)aryl, (C₁-C₉)heterocycloalkyl or (C₁-C₉)heteroaryl is optionally independently substituted with one or more substituents independently selected from the group consisting of halogen, hydroxy, cyano, nitro, amino, (C₁-C₆)alkylamino, di(C₁-C₆)alkylamino, (C₁-C₆)alkyl optionally substituted with one or more halogen or (C₁-C₆)alkoxy or (C₆-C₁₀)aryloxy, (C₆-C₁₀)aryl optionally substituted with one or more halogen or (C₁-C₆)alkoxy or (C₁-C₆)alkyl or trihalo(C₁-C₆)alkyl, (C₁-C₉)heterocycloalkyl optionally substituted with (C₆-C₁₀)aryl or (C₁-C₉)heteroaryl or ═O or alkyl optionally substituted with hydroxy, (C₃-C₁₀)cycloalkyl optionally substituted with hydroxy, (C₁-C₉)heteroaryl optionally substituted with one or more halogen or (C₁-C₆)alkoxy or (C₁-C₆)alkyl or trihalo(C₁-C₆)alkyl, halo(C₁-C₆)alkyl, hydroxy(C₁-C₆)alkyl, carboxy, (C₁-C₆)alkoxy, (C₆-C₁₀)aryloxy, (C₁-C₆)alkoxycarbonyl, aminocarbonyl, (C₁-C₆)alkylaminocarbonyl and di(C₁-C₆)alkylaminocarbonyl;

R¹⁰ is independently selected from the group consisting of hydrogen, —CN, halogen, —(C═O)R⁹, —(C═O)NR⁹, NR⁹, —OR⁹ or —R⁹;

ring “A” is (C₆-C₁₀)aryl, (C₁-C₉)heteroaryl, (C₄-C₁₀)cycloalkyl, or (C₁-C₉)heterocycloalkyl;

“X” is >NH, —O— or >C(R⁴)₂; and

“Y” is absent, >NR¹¹, —NR¹¹—(C═O)—, —O— or >C(R⁷)₂.

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like.

In some instances, the number of carbon atoms in a hydrocarbyl substituent (e.g., alkyl, alkenyl, cycloalkyl, cycloalkenyl, aryl, etc.) is indicated by the prefix “Cₓ-C_{y}—,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl refers to saturated cycloalkyl containing from 3 to 6 carbon ring atoms.

The term “hydrogen” refers to hydrogen substituent, and may be depicted as —H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “cyano” (also referred to as “nitrile”) means —CN, which also may be depicted:

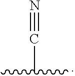

The term “carbonyl” means —C(O)—, which also may be depicted as:

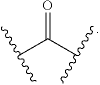

The term “amino” refers to —NH₂.

The term “oxo” refers to ═O.

The term “alkoxy” refers to an alkyl linked to an oxygen, which may also be represented as:

—O—R, wherein the R represents the alkyl group. Examples of alkoxy include methoxy, ethoxy, propoxy and butoxy.

The term “sulfonyl” refers to —S(O)₂—, which also may be depicted as:

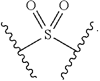

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

As used herein, the term “aryl” is defined to include all-carbon monocyclic or fused-ring polycyclic (i.e., rings which share adjacent pairs of carbon atoms) groups having a completely conjugated pi-electron system. The aryl group has 6, 8, 9, 10 or 12 carbon atoms in the ring(s). Preferably, the aryl group has 6, 8, 9 or 10 carbon atoms in the ring(s). More preferably, the aryl group has 6 or 10 carbon atoms in the ring(s). Most preferably, the aryl group has 6 carbon atoms in the ring(s). For example, as used herein, the term “(C₆-C₁₀)aryl” means aromatic radicals containing from 6 to 10 carbon atoms such as phenyl, naphthyl, tetrahydronaphthyl, anthracenyl, indanyl and the like. The aryl group is optionally substituted by 1 to 5 suitable substituents.

As used herein, the term “heteroaryl” is defined to include monocyclic or fused-ring polycyclic aromatic heterocyclic groups with one or more heteroatoms selected from O, S and N in the ring. The heteroaryl group has 5 to 12 ring atoms including one to five heteroatoms selected from O, S, and N. Preferably, the heteroaryl group has 5 to 10 ring atoms including one to four heteroatoms. More preferably, the heteroaryl group has 5 to 8 ring atoms including one, two or three heteroatoms. Most preferably, the heteroaryl group has 6 to 8 ring atoms including one or two heteroatoms. For example, as used herein, the term “5 to 12 membered heteroaryl” means aromatic radicals containing at least one ring heteroatom selected from O, S and N and from 1 to 11 carbon atoms such as pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like. The heteroaryl group is optionally substituted by 1 to 5 suitable substituents.

As used herein, the term “heterocycloalkyl” is defined to include a monocyclic, bridged, polycyclic or fused polycyclic saturated or unsaturated non-aromatic 3 to 20 membered ring including 1 or more heteroatoms selected from O, S and N. Examples of such heterocycloalkyl rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, and the like. Further examples of said heterocycloalkyl rings are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2 pyrazolidin-2-yl, 1,3-pyrazolidin-1-yl, 1,2-tetrahydrothiazin-2-yl, 1,3 tetrahydrothiazin-3-yl, 1,2-tetrahydrodiazin-2-yl, 1,3 tetrahydrodiazin-1-yl, 1,4-oxazin-2-yl, 1,2,5-oxathiazin-4-yl and the like. The heterocycloalkyl ring is optionally substituted by 1 to 5 suitable substituents.

If substituents are described as being “independently selected” from a group, each substituent is selected independent of the other. Each substituent therefore may be identical to or different from the other substituent(s).

When an asymmetric center is present in a compound of formula I (hereinafter understood to mean formula I, Ia, Ib, or Ic), hereinafter referred to as a “compound of the invention,” the compound may exist in the form of optical isomers (enantiomers). In one embodiment, the present invention comprises enantiomers and mixtures, including racemic mixtures of the compounds of formula I. In another embodiment, for compounds of formula I that contain more than one asymmetric center, the present invention comprises diastereomeric forms (individual diastereomers and mixtures thereof) of compounds. When a compound of formula I contains an alkenyl group or moiety, geometric isomers may arise.

The present invention comprises the tautomeric forms of compounds of formula I. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

In one embodiment, hemisalts of acids and bases may also be formed, for example, hemisulphate and hemicalcium salts.

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³⁵S, ¹⁸F, and Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

An embodiment of the present invention relates to a compound of the Formula:

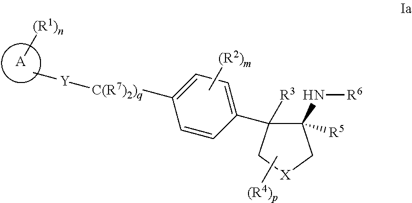

Another embodiment of the present invention relates to a compound of the Formula:

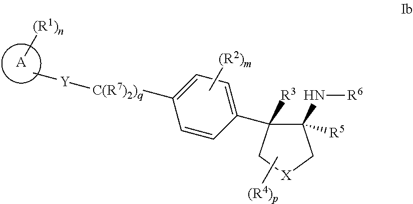

Another embodiment of the present invention relates to a compound of the Formula

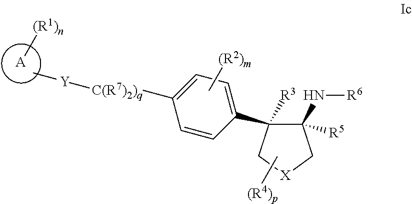

Another embodiment of the present invention relates to a compound of the Formula I (or Ia, Ib or Ic), wherein X is —O—.

Another embodiment of the present invention relates to a compound of the Formula I (or Ia, Ib or Ic), wherein X is >NH.

Another embodiment of the present invention relates to a compound of the Formula I (or Ia, Ib or Ic), wherein X is >0(R⁴)₂.

Another embodiment of the present invention relates to a compound of the Formula I (or Ia, Ib or Ic), wherein X is >0(R⁴)₂ and each R⁴ is hydrogen.

Another embodiment of the present invention relates to a compound of the Formula I (or Ia, Ib or Ic), wherein ring A is phenyl and R¹ is in the ortho position relative to Y.

Another embodiment of the present invention relates to a compound of the Formula I (or Ia, Ib or Ic), wherein ring “A” is (C₁-C₉)heteroaryl (more specifically thienyl, furyl, imidazolyl, pyrrolyl, oxazolyl, thiazolyl, pyrazolyl, tetrazolyl, triazolyl, oxadiazolyl, or thiadiazolyl; n is one; and wherein R¹ is hydrogen, halogen, hydroxyl, (C₁-C₆)alkoxy, cyano, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, or (C₁-C₆)alkyl-S(O)₂—NH—, wherein said (C₁-C₆)alkoxy, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl.

Another embodiment of the present invention relates to a compound of the Formula I (or Ia, Ib or Ic), wherein ring “A” is (C₁-C₉)heterocycloalkyl (more specifically azetidinyl, tetrahydrofuranyl, imidazolidinyl, pyrrolidinyl, oxazolidinyl, thiazolidinyl, tetrahydrothiazinyl, tetrahydro-thiadiazinyl, oxetanyl, or tetrahydrodiazinyl); n is one; and wherein R¹ is hydrogen, halogen, hydroxyl, (C₁-C₆)alkoxy, cyano, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, or (C₁-C₆)alkyl-S(O)₂—NH—, wherein said (C₁-C₆)alkoxy, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl.

Another embodiment of the present invention relates to a compound of the Formula I (or Ia, Ib or Ic), wherein ring A is phenyl; n is one; R¹ is in the ortho position relative to Y; and wherein R¹ is hydrogen, halogen, hydroxyl, (C₁-C₆)alkoxy, cyano, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, or (C₁-C₆)alkyl-S(O)₂—NH—, wherein said (C₁-C₆)alkoxy, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl.

Another embodiment of the present invention relates to a compound of the Formula I (or Ia, Ib or Ic), wherein R¹ is (C₁-C₆)alkoxy (more specifically methoxy and ethoxy), (C₁-C₆)alkyl (more specifically methyl and ethyl), cyano or halogen and is in the ortho or para position relative to Y.

Another embodiment of the present invention relates to a compound of the Formula I (or Ia, Ib or Ic), wherein R² is hydrogen.

Another embodiment of the present invention relates to a compound of the Formula I (or Ia, Ib or Ic), wherein R⁴ is hydrogen.

Another embodiment of the present invention relates to a compound of the Formula I (or Ia, Ib or Ic), wherein p is two and both R⁴ are taken together to form oxo.

Another embodiment of the present invention relates to a compound of the Formula I (or Ia, Ib or Ic), wherein p is two and each R⁴ is (C₁-C₆)alkoxy.

Another embodiment of the present invention relates to a compound of the Formula I (or Ia, Ib or Ic), wherein q is zero.

Another embodiment of the present invention relates to a compound of the Formula I (or Ia, Ib or Ic), wherein Y is absent.

Yet other embodiments of the present invention relate to so called amidotetrahydrofurans of Formula I (and Ia, Ib or Ic) wherein R⁶ is (C₁-C₅)alkyl-(C═O)—.

Yet other embodiments of the present invention relate to so called uredotetrahydrofurans of Formula I (and Ia, Ib, or Ic) wherein R⁶ is [(C₁-C₆)alkyl]₂N—(C═O)—, wherein said (C₁-C₆)alkyl moieties (more preferably one to two carbon atoms) may optionally be taken together with the nitrogen atom to which they are attached to form a four to six membered heterocyclic ring.

Yet other embodiments of the present invention relate to alkylsulfonyltetrahydrofurans of Formula I (and Ia, Ib, or Ic) wherein R⁶ is (C₁-C₆)alkyl-SO₂— (more preferably one to two carbon atoms).

Yet other embodiments of the present invention relate to cycloalkylsulonyltetrahydrofurans of Formula I (and Ia, Ib, or Ic) wherein R⁶ is (C₃-C₆)cycloalkyl-SO₂—.

Yet other embodiments of the present invention relate to sulfonamidotetrahydrofurans of Formula I (and Ia, Ib, or Ic) wherein R⁶ is [(C₁-C₆)alkyl]₂N—SO₂—; wherein said (C₁-C₆)alkyl moieties (more preferably one to two carbon atoms) may optionally be taken together with the nitrogen atom to which they are attached to form a four to six membered heterocyclic ring.

Specific preferred compounds of the invention include:
N-{(3S,4S)-4-[4-(5-cyano-2-thienyl)phenyl]-4-hydroxytetrahydrofuran-3-yl}propane-2-sulfonamide; and
N-[(3S,4S)-4-biphenyl-4-yl-4-hydroxytetrahydrofuran-3-yl]propane-2-sulfonamide or pharmaceutically acceptable salts thereof.

Other specific compounds of the invention, and the pharmaceutically acceptable salts thereof, include the following:
N-{(3R,4R)-4-[4-(5-cyano-2-thienyl)phenyl]-4-hydroxytetrahydrofuran-3-yl}propane-2-sulfonamide;
N-[(3R,4R)-4-biphenyl-4-yl-4-hydroxytetrahydrofuran-3-yl]propane-2-sulfonamide;
[4-(2′-cyano-biphenyl-4-yl)-4-hydroxy-tetrahydro-furan-3-yl]propane-2-sulfonamide;
N-[4-(2′-cyano-biphenyl-4-yl)-4-hydroxy-tetrahydro-furan-3-yl]-isobutyramide;

N′-[4-(2′-cyano-biphenyl-4-yl)-4-hydroxy-tetrahydro-furan-3-yl]-N,N-dimethylsulfamide;
[4-(2′-cyano-4′-fluoro-biphenyl-4-yl)-4-hydroxy-tetrahydro-furan-3-yl]propane-2-sulfonamide;
2-Cyano-4′-[3-hydroxy-4-(propane-2-sulfonylamino)-tetrahydro-furan-3-yl]-biphenyl-4-carboxylic acid;

[4-(3′-cyano-biphenyl-4-yl)-4-hydroxy-tetrahydro-furan-3-yl]propane-2-sulfonamide;
[4-(4′-cyano-biphenyl-4-yl)-4-hydroxy-tetrahydro-furan-3-yl]propane-2-sulfonamide;
[4-(2′-methyl-biphenyl-4-yl)-4-hydroxy-tetrahydro-furan-3-yl]propane-2-sulfonamide;
[4-(4′-methyl-biphenyl-4-yl)-4-hydroxy-tetrahydro-furan-3-yl]propane-2-sulfonamide;
[4-(2′-fluoro-biphenyl-4-yl)-4-hydroxy-tetrahydro-furan-3-yl]propane-2-sulfonamide;
[4-(4′-fluoro-biphenyl-4-yl)-4-hydroxy-tetrahydro-furan-3-yl]propane-2-sulfonamide;
[4-(2′-chloro-biphenyl-4-yl)-4-hydroxy-tetrahydro-furan-3-yl]propane-2-sulfonamide;
[4-(4′-chloro-biphenyl-4-yl)-4-hydroxy-tetrahydro-furan-3-yl]propane-2-sulfonamide;
[4-(Z-hydroxy-biphenyl-4-yl)-4-hydroxy-tetrahydro-furan-3-yl]propane-2-sulfonamide;
[4-(4′-hydroxy-biphenyl-4-yl)-4-hydroxy-tetrahydro-furan-3-yl]propane-2-sulfonamide;
[4-(2′-methoxy-biphenyl-4-yl)-4-hydroxy-tetrahydro-furan-3-yl]propane-2-sulfonamide;
[4-(2′-ethoxy-biphenyl-4-yl)-4-hydroxy-tetrahydro-furan-3-yl]propane-2-sulfonamide;
{4-[2′-(2,2,2-trifluoro-ethoxy)-biphenyl-4-yl]-4-hydroxy-tetrahydro-furan-3-yl}propane-2-sulfonamide;
4′-[3-Hydroxy-4-(propane-2-sulfonylamino)-tetrahydro-furan-3-yl]-biphenyl-2-carboxamide;
{4-hydroxy-4-[2′-(pyrrolidine-1-sulfonyl)-biphenyl-4-yl]-tetrahydro-furan-3-yl}propane-2-sulfonamide;
[4-(2′-methanesulfonylamino-biphenyl-4-yl)-4-hydroxy-tetrahydro-furan-3-yl]propane-2-sulfonamide;
[4-(2′-methoxymethyl-biphenyl-4-yl)-4-hydroxy-tetrahydro-furan-3-yl]propane-2-sulfonamide;
N-{4′-[3-Hydroxy-4-(propane-2-sulfonylamino)-tetrahydro-furan-3-yl]-biphenyl-4-yl}-acetamide;
{4-hydroxy-4-[4-(4-methyl-thiophen-2-yl)-phenyl]-tetrahydro-furan-3-yl}propane-2-sulfonamide;
[4-hydroxy-4-(4-pyridin-2-yl-phenyl)-tetrahydro-furan-3-yl]propane-2-sulfonamide;
[4-hydroxy-4-(4-pyridin-3-yl-phenyl)-tetrahydro-furan-3-yl]propane-2-sulfonamide;
[4-hydroxy-4-(4-pyridin-4-yl-phenyl)-tetrahydro-furan-3-yl]propane-2-sulfonamide;
[4-hydroxy-4-(4-pyrimidin-5-yl-phenyl)-tetrahydro-furan-3-yl]propane-2-sulfonamide;
[4-hydroxy-4-(4-pyrrolidin-1-yl-phenyl)-tetrahydro-furan-3-yl]propane-2-sulfonamide;
N-(1-{4-[3-Hydroxy-4-(propane-2-sulfonylamino)-tetrahydro-furan-3-yl]-phenyl}-pyrrolidin-3-yl)-acetamide;
[4-hydroxy-4-(4-phenoxy-phenyl)-tetrahydro-furan-3-yl]propane-2-sulfonamide.

The compounds of Formula I are useful for the treatment of a variety of neurological and psychiatric disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, attention deficit disorder, and conduct disorder. Accordingly, in one embodiment, the invention provides a method for treating a condition in a mammal, such as a human, selected from the conditions above, comprising administering a compound of Formula I to the mammal. The mammal is preferably a mammal in need of such treatment or prevention.

The term “treating”, as used herein, unless otherwise indicated, means reversing, alleviating, modulating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

As an example, the invention provides a method for treating a condition selected from migraine, anxiety disorders, schizophrenia, and epilepsy. Exemplary anxiety disorders are generalized anxiety disorder, social anxiety disorder, panic disorder, post-traumatic stress disorder and obsessive-compulsive disorder. As another example, the invention provides a method for treating depression selected from Major Depression, Chronic Depression (Dysthymia), Seasonal Depression (Seasonal Affective Disorder), Psychotic Depression, and Postpartum Depression. As another example, the invention provides a method for treating a sleep disorder selected from insomnia and sleep deprivation.

In another embodiment, the invention comprises methods of treating a condition in a mammal, such as a human, by administering a compound of Formula I, wherein the condition is selected from the group consisting of atherosclerotic cardiovascular diseases, cerebrovascular diseases and peripheral arterial diseases, to the mammal. The mammal is preferably a mammal in need of such treatment or prevention. Other conditions that can be treated in accordance with the present invention include hypertension and angiogenesis.

In another embodiment the present invention provides methods of treating neurological and psychiatric disorders associated with glutamate dysfunction, comprising administering to a mammal, preferably a mammal in need thereof, an amount of a compound of Formula I effective in treating such disorders.

The compound of Formula I is optionally used in combination with another active agent. Such an active agent may be, for example, an atypical antipsychotic or an AMPA potentiator. Accordingly, another embodiment of the invention provides methods of treating neurological and psychiatric disorders associated with glutamate dysfunction, comprising administering to a mammal an amount of a compound of Formula I and further comprising administering another active agent.

As used herein, the term “another active agent” refers to any therapeutic agent, other than the compound of Formula (I), or salt thereof, that is useful for the treatment of a subject disorder. Examples of additional therapeutic agents include antidepressants, antipsychotics, anti-pain and anti-anxiety agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, a-adrenoreceptor antagonists, and atypical antidepressants. Suitable norepinephrine reuptake inhibitors include tertiary amine tricyclics and secondary amine tricyclics. Examples of suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Examples of suitable selective serotonin reuptake inhibitors include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Example of suitable serotonin and noradrenaline reuptake inhibitors of use in the present invention include venlafaxine. Examples of suitable atypical anti-depressants include bupropion, lithium, nefazodone, trazodone and viloxazine. Examples of suitable classes of anti-anxiety agents that can be used in combination with the compounds of the invention include benzodiazepines and serotonin 1A (5-HT1A) agonists or antagonists, especially 5-HT1A partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT1A receptor agonists or antagonists include buspirone, flesinoxan, gepirone and ipsapirone. Suitable atypical antipsychotics include paliperidone, bifeprunox, ziprasidone, risperidone, aripiprazole, olanzapine, and quetiapine. Suitable nicotine acetylcholine agonists include ispronicline, varenicline and MEM 3454. Anti-pain agents include pregabalin, gabapentin, clonidine, neostigmine, baclofen, midazolam, ketamine and ziconotide.

The invention is also directed to a pharmaceutical composition comprising a compound of Formula I, and a pharmaceutically acceptable carrier.

### DETAILED DESCRIPTION OF THE INVENTION

The compounds of the Formula I may be prepared by the methods described below, together with synthetic methods known in the art of organic chemistry, or modifications and derivatisations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1999, which is hereby incorporated by reference.

As appreciated by the artisan, the use of Formula I is a convenience and the invention is understood to include each and every species falling thereunder as though individually set forth herein. Thus the invention contemplates each species separately and any and all combinations of such species.

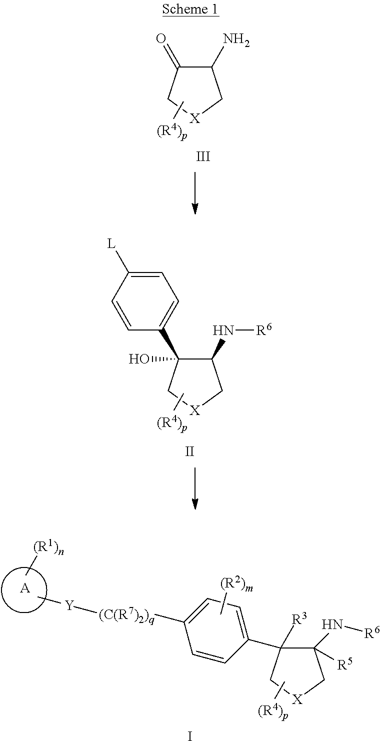

Scheme 1 refers to the preparation of compounds of the Formula I. Referring to Scheme 1, an aryl halide of Formula II, wherein L is iodo, bromo or a triflate, can be coupled to a suitably substituted aryl boronic acid of structure (R¹)ₙ-ArB(OH)₂, wherein Ar represents a suitably substituted aryl or heteroaryl group, under standard palladium catalyzed cross-coupling reaction conditions well known to one of ordinary skill in the art to provide the compound of Formula I. [Suzuki, A., Journal of Organometallic Chemistry, 576, 147-169 (1999), Miyaura and Suzuki, Chemical Reviews, 95, 2457-2483 (1995).] More specifically, the aryl iodinate, bromate or triflate of Formula III is combined with 1 to 3 equivalents of aryl boronic acid and a suitable base, such as 2 to 5 equivalents of potassium carbonate, in a suitable organic solvent such as THF. A palladium catalyst is added, such as 0.02 equivalents of palladium tetrakistriphenylphosphine, and the reaction mixture is heated to temperatures ranging from 60 to 100° C. for 1 to 24 hours. The reaction is not limited to the employment of this solvent, base, or catalyst as many other conditions may be used.

Alternatively, a compound of Formula I can be prepared from a compound of Formula II, wherein “L” is a silyl group (such as trimethylsilyl) by first converting the silyl group to a halide, such as by reaction with a halogenating reagent such as potassium bromide/N-Chlorosuccinimide (NCS) in the presence of an acid (such as acetic acid) followed by arylation as described above. Suitable solvents for the halogenation include alcohols such as methanol or ethanol. The reaction can be conducted at a temperature of about 10° C. to about 60° C. for about 10 to about 120 minutes.

Alternatively, a compound of Formula I wherein q is zero and Y is O or NR⁷ can be prepared by reaction of a compound of Formula II wherein L is NH₂ or OH by reaction with an aryl halide in the presence of a catalyst.

Alternatively, when q is two or three, one skilled in the art will appreciate that numerous coupling reactions of two suitably functionalized alkyl groups can afford the compounds of Formula I. Such reactions are within the skill of the art.

The compound of Formula II can be prepared from a compound of Formula III by coupling with a suitably substituted Aryl Grignard in an ethereal solvent such as THF at about −30° C. to about room temperature. A catalyst, such as palladium or copper can facilitate the reaction.

The compounds of Formula III are commercially available or can be made by methods well known to those skilled in the art.

The compounds of Formula I can be separated into the enantiomerically pure isomers according to methods well known to those skilled in the art and described in detail in the Example section herein.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

### Administration and Dosing

Typically, a compound of the invention is administered in an amount effective to treat or prevent a condition as described herein. The compounds of the invention are administered by any suitable route in the form of a pharmaceutical composition adapted to such a route, and in a dose effective for the treatment or prevention intended. Therapeutically effective doses of the compounds required to treat or prevent the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment or prevention of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment or prevention of the conditions recited herein.

### Pharmaceutical Compositions

For the treatment or prevention of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment or prevention intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

### Co-Administration

The compounds of the present invention can be used, alone or in combination with other therapeutic agents, in the treatment or prevention of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment or prevention described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### EXPERIMENTAL PROCEDURES

Experiments were generally carried out under inert atmosphere (nitrogen or argon) particularly in cases where oxygen or moisture sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Chemical shifts for nuclear magnetic resonance (NMR) data are expressed in parts per million (ppm, δ) referenced to residual peaks from the deuterated solvents employed.

Single Crystal X-Ray Analysis

A representative crystal was surveyed and a 0.90 Å data set (maximum sin Θ/λ=0.56) was collected on a Bruker APEX diffractometer. Friedel pairs were collected in order to facilitate the determination of the absolute configuration. Atomic scattering factors were taken from the International Tables for Crystallography. All crystallographic calculations were facilitated by the SHELXTL (SHELXTL, Version 5.1, Bruker AXS, 1997) system. All diffractometer data were collected at room temperature. Pertinent crystal, data collection, and refinement are summarized in tables accompanying each example.

A trial structure was obtained by direct methods. This trial structure refined routinely. Hydrogen positions were calculated wherever possible. The methyl hydrogens were located by difference Fourier techniques and then idealized. The hydrogen on nitrogen was located by difference Fourier techniques and allowed to refine. The hydrogen parameters were added to the structure factor calculations but were not refined. The shifts calculated in the final cycles of least squares refinement were all less than 0.1 of the corresponding standard deviations. The final R-index was 3.95%. A final difference Fourier revealed no missing or misplaced electron density. The refined structure was plotted using the SHELXTL plotting package.

The absolute configuration was determined by the method of Flack (Acta Crystallogr., A39, 876, 1983). Coordinates, anisotropic temperature factors, distances and angles are included with the relevant examples as supplementary material.

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification. Chemical shifts for nuclear magnetic resonance (NMR) data are expressed in parts per million (ppm, δ) referenced to residual peaks from the deuterated solvents employed.

### Preparation 1

### N-(4-Oxotetrahydrofuran-3-yl)propane-2-sulfonamide

### Scheme 1

Step 1. Preparation of N-(4-hydroxytetrahydrofuran-3-yl)propane-2-sulfonamide.

A solution of 3,6-dioxabicyclo[3.1.0]hexane (10.3 g, 120 mmol) in 1,4-dioxane (30 mL) was treated with propane-2-sulfonamide (17.7 g, 144 mmol), benzyltriethylammonium chloride (2.72 g, 12.0 mmol) and potassium carbonate (1.65 g, 12 mmol), and the mixture was heated at 90° C. for 5 days. The reaction mixture was then filtered, concentrated in vacuo and purified via silica gel chromatography (Gradient: 20% to 60% ethyl acetate in heptane) to afford N-(4-hydroxytetrahydrofuran-3-yl)propane-2-sulfonamide. Yield: 18.5 g, 88.4 mmol, 74%. LCMS m/z 210.1 (M+1). ¹H NMR (400 MHz, CDCl₃) δ 1.37 (d, J=6.6 Hz, 3H), 1.39 (d, J=6.6 Hz, 3H), 3.21 (septet, J=6.7 Hz, 1H), 3.69 (m, 2H), 3.81 (m, 1H), 4.09 (m, 2H), 4.38 (m, 1H), 4.97 (d, J=8.3 Hz, 1H). ¹³C NMR (100 MHz, CDCl₃) δ 16.41, 16.65, 54.11, 61.93, 71.45, 73.46, 77.51.

Step 2. Preparation of N-(4-oxotetrahydrofuran-3-yl)propane-2-sulfonamide.

A solution of oxalyl chloride (3.13 mL, 35.9 mmol) in dichloromethane (75 mL) was cooled to −78° C. and treated with dimethyl sulfoxide (3.8 mL, 53 mmol). After 5 minutes, a solution of N-(4-hydroxytetrahydrofuran-3-yl)propane-2-sulfonamide (5.0 g, 24 mmol) in dichloromethane was added, and the reaction was stirred at −78° C. for an additional 15 minutes. Triethylamine (16 mL, 115 mmol) was added, and the reaction was allowed to warm to room temperature and stir for 18 hours. Water (100 mL) was added, and the layers were separated. After extraction of the aqueous layer with dichloromethane (2×50 mL), the combined organic layers were dried over sodium sulfate, filtered and concentrated under reduced pressure. The residue was purified by silica gel chromatography (Gradient: 20% to 50% ethyl acetate in heptane) to provide N-(4-oxotetrahydrofuran-3-yl)propane-2-sulfonamide as an oil. Yield: 2.0 g, 9.6 mmol, 40%. LCMS m/z 208.1 (M+1). ¹H NMR (400 MHz, CDCl₃) δ 1.26 (d, J=6.8 Hz, 3H), 1.27 (d, J=6.6 Hz, 3H), 3.16 (septet, J=6.8 Hz, 1H), 3.66 (dd, J=10.1, 9.2 Hz, 1H), 3.79 (d, J=17.6 Hz, 1H), 4.06 (d, J=17.4 Hz, 1H), 4.11 (m, 1H), 4.44 (dd, J=8.9, 8.9 Hz, 1H), 5.46 (d, J=7.5 Hz, 1H).

### Preparation 2

### [4-(trimethylsilyl)phenyl]magnesium bromide

Magnesium (0.583 g, 24.0 mmol) was added to a solution of (4-bromophenyl)(trimethyl)silane (5 g, 20 mmol) and iodine (6 mg, 0.02 mmol) in tetrahydrofuran (40 mL), and the reaction was stirred at room temperature for 2 hours. The suspension was then heated to reflux for 2.5 hours, until almost all of the magnesium had been consumed. The solution was cooled to room temperature to provide a 0.5 M solution of the title compound in tetrahydrofuran.

### Examples 1 and 2

### N-{(3S,4S)-4-[4-(5-cyano-2-thienyl)phenyl]-4-hydroxytetrahydrofuran-3-yl}propane-2-sulfonamide and N-{(3R,4R)-4-[4-(5-cyano-2-thienyl)phenyl]-4-hydroxytetrahydrofuran-3-yl}propane-2-sulfonamide

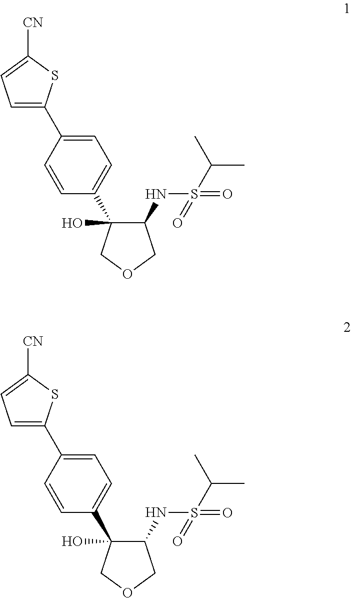

Step 1. Preparation of trans-N-{4-hydroxy-4-[4-(trimethylsilyl)phenyl]tetrahydrofuran-3-yl}propane-2-sulfonamide and cis-N-{4-hydroxy-4-[4-(trimethylsilyl) phenyl]tetrahydrofuran-3-yl}propane-2-sulfonamide.

A solution of N-(4-oxotetrahydrofuran-3-yl)propane-2-sulfonamide (0.43 g, 2.1 mmol) in tetrahydrofuran (5 mL) at 0° C. was treated with [4-(trimethylsilyl)phenyl]magnesium bromide (0.5 M solution in tetrahydrofuran, 8.3 mL, 4.15 mmol), and stirred at 0° C. for 4 hours, then at room temperature for 66 hours. The reaction mixture was then recooled to 0° C. and quenched with saturated aqueous ammonium chloride solution. The layers were separated and the aqueous layer was extracted with ethyl acetate (2×20 mL). The organic layers were combined with those of two similar reactions with identical procedures (N-(4-oxotetrahydrofuran-3-yl)propane-2-sulfonamide used: 0.75 g, 3.6 mmol), dried over sodium sulfate, concentrated in vacuo and subjected to purification via silica gel chromatography (Gradient 0-10% acetone in dichloromethane). This afforded a mixture of trans-N-{4-hydroxy-4-[4-(trimethylsilyl)phenyl]tetrahydrofuran-3-yl}propane-2-sulfonamide and cis-N-{4-hydroxy-4-[4-(trimethylsilyl)phenyl]tetrahydrofuran-3-yl}propane-2-sulfonamide as a yellow oil, with cis-N-{4-hydroxy-4-[4-(trimethylsilyl)phenyl]tetrahydrofuran-3-yl}propane-2-sulfonamide as the major isomer. Yield: 350 mg, <0.98 mmol, <17%. LCMS m/z 356.1 (M−1). ¹H NMR (400 MHz, CDCl₃) Selected signals from major isomer cis-N-{4-hydroxy-4-[4-(trimethylsilyl)phenyl]tetrahydrofuran-3-yl}propane-2-sulfonamide: δ 1.04 (d, J=6.8 Hz, 3H), 1.18 (d, J=6.8 Hz, 3H), 3.71 (dd, J=9.4, 5.9 Hz, 1H), 4.03 (d, J=9.5 Hz, 1H), 4.31 (d, J=9.5 Hz, 1H), 4.36 (dd, J=9.3, 6.6 Hz, 1H), 7.48 (m, 2H), 7.55 (m, 2H). The relative stereochemistry of the major isomer cis-N-{4-hydroxy-4-[4-(trimethylsilyl)phenyl]tetrahydrofuran-3-yl}propane-2-sulfonamide was assigned on the basis of literature work; see L. E. Overman, M. E. Okazaki and P. Mishra, Tetrahedron Letters 1986, 27, 4391-4394.

Step 4. Preparation of trans-N-[4-(4-bromophenyl)-4-hydroxytetrahydrofuran-3-yl]propane-2-sulfonamide and cis-N-[4-(4-bromophenyl)-4-hydroxytetrahydrofuran-3-yl]propane-2-sulfonamide.

The mixture of trans-N-{4-hydroxy-4-[4-(trimethylsilyl)phenyl]tetrahydrofuran-3-yl}propane-2-sulfonamide and cis-N-{4-hydroxy-4-[4-(trimethylsilyl)phenyl]tetrahydrofuran-3-yl}propane-2-sulfonamide prepared in the preceding step (0.3 g, 0.8 mmol) was combined with potassium bromide (150 mg, 1.26 mmol) in acetic acid (5.6 mL) and methanol (1 mL), and the mixture was stirred at 60° C. for 20 minutes. N-chlorosuccinimide (134 mg, 1.0 mmol) was added, and the reaction was stirred at 60° C. for an additional 4 hours, then cooled to room temperature and stirred for 66 hours. The reaction was poured onto a mixture of sodium hydroxide (7 g) and ice (30 g). The resulting solution was extracted with ethyl acetate (3×20 mL), and the combined organic layers were dried over sodium sulfate, filtered and concentrated in vacuo to provide a mixture of trans-N-[4-(4-bromophenyl)-4-hydroxytetrahydrofuran-3-yl]propane-2-sulfonamide and cis-N-[4-(4-bromophenyl)-4-hydroxytetrahydrofuran-3-yl]propane-2-sulfonamide, highly enriched in cis-N-[4-(4-bromophenyl)-4-hydroxytetrahydrofuran-3-yl]propane-2-sulfonamide. This was used in the next step without purification. Yield: 0.2 g, 0.5 mmol, 62%. LCMS m/z 361.9 (M−1). ¹H NMR (400 MHz, CDCl₃) Selected signals from major product cis-N-[4-(4-bromophenyl)-4-hydroxytetrahydrofuran-3-yl]propane-2-sulfonamide: δ 1.15 (d, J=6.8 Hz, 3H), 1.22 (d, J=6.8 Hz, 3H), 3.66 (dd, J=9.3, 6.8 Hz, 1H), 4.04 (d, J=9.5 Hz, 1H), 4.17 (m, 1H), 4.25 (d, J=9.3 Hz, 1H), 4.31 (br d, J=9.8 Hz, 1H), 4.36 (dd, J=9.3, 7.3 Hz, 1H), 4.83 (br s, 1H), 7.42 (m, 2H), 7.53 (m, 2H).

Step 5. Preparation of trans-N-{4-[4-(5-cyano-2-thienyl)phenyl]-4-hydroxytetrahydrofuran-3-yl}propane-2-sulfonamide and cis-N-{4-[4-(5-cyano-2-thienyl)phenyl]-4-hydroxytetrahydrofuran-3-yl}propane-2-sulfonamide.

A 2 mL microwave vial was charged with the mixture of trans-N-[4-(4-bromophenyl)-4-hydroxytetrahydrofuran-3-yl]propane-2-sulfonamide and cis-N-[4-(4-bromophenyl)-4-hydroxytetrahydrofuran-3-yl]propane-2-sulfonamide prepared in the preceding step (0.2 g, 0.5 mmol), (5-cyano-2-thienyl)boronic acid (128 mg, 0.837 mmol), dicyclohexyl(2′,4′,6′-triisopropylbiphenyl-2-yl)phosphine (26.2 mg, 0.055 mmol), palladium(II) acetate (8.1 mg, 0.036 mmol), potassium fluoride (160 mg, 2.7 mmol), toluene (1 mL) and methanol (1 mL). The vial was capped, the contents degassed, and the reaction was subjected to microwave irradiation for 35 minutes at 130° C. Removal of solvent in vacuo was followed by partitioning of the residue between ethyl acetate and saturated aqueous sodium chloride solution. The aqueous layer was extracted twice with ethyl acetate and the combined organic layers were dried over sodium sulfate. Filtration, removal of solvent in vacuo and purification via silica gel chromatography (Gradient: 20% to 50% ethyl acetate in heptane) provided a mixture of trans-N-{4-[4-(5-cyano-2-thienyl)phenyl]-4-hydroxytetrahydrofuran-3-yl}propane-2-sulfonamide and cis-N-{4-[4-(5-cyano-2-thienyl)phenyl]-4-hydroxytetrahydrofuran-3-yl}propane-2-sulfonamide as an oil, highly enriched in cis-N-{4-[4-(5-cyano-2-thienyl)phenyl]-4-hydroxytetrahydrofuran-3-yl}propane-2-sulfonamide. Yield: 35 mg, 0.089 mmol, 18%. LCMS m/z 390.9 (M+1).

Step 6. Isolation of N-{(3S,4S)-4-[4-(5-cyano-2-thienyl)phenyl]-4-hydroxytetrahydrofuran-3-yl}propane-2-sulfonamide and N-{(3R,4R)-4-[4-(5-cyano-2-thienyl)phenyl]-4-hydroxytetrahydrofuran-3-yl}propane-2-sulfonamide.

The mixture of trans-N-{4-[4-(5-cyano-2-thienyl)phenyl]-4-hydroxytetrahydrofuran-3-yl}propane-2-sulfonamide and cis-N-{4-[4-(5-cyano-2-thienyl)phenyl]-4-hydroxytetrahydrofuran-3-yl}propane-2-sulfonamide isolated in the previous step (35 mg, 0.089 mmol) was subjected to chiral chromatography using a Chiralcel OJ-H column, 5 uM, 1 cm×25 cm (Mobile phase: 75:25:0.2 carbon dioxide:methanol:isopropylamine; Flow rate: 10 g/min).

Material eluting at 4.90 minutes was collected to yield N-{(3S,4S)-4-[4-(5-cyano-2-thienyl)phenyl]-4-hydroxytetrahydrofuran-3-yl}propane-2-sulfonamide as a solid. Yield: 9.7 mg, 0.025 mmol. LCMS m/z 391.1 (M−1). ¹H NMR (400 MHz, CDCl₃) δ 1.26 (d, J=6.8 Hz, 3H), 1.30 (d, J=6.8 Hz, 3H), 3.08 (septet, J=6.8 Hz, 1H), 3.61 (br s, 1H), 3.66 (dd, J=9.3, 7.7 Hz, 1H), 3.77 (d, J=10.2 Hz, 1H), 4.13 (d, J=9.5 Hz, 1H), 4.26 (m, 1H), 4.34 (d, J=9.5 Hz, 1H), 4.41 (dd, J=9.3, 7.7 Hz, 1H), 7.32 (d, J=3.9 Hz, 1H), 7.61 (d, J=3.9 Hz, 1H), 7.66 (m, 4H).

Material eluting at 3.38 minutes was collected to yield N-{(3R,4R)-4-[4-(5-cyano-2-thienyl)phenyl]-4-hydroxytetrahydrofuran-3-yl}propane-2-sulfonamide as a solid. Yield: 11 mg, 0.028 mmol. LCMS m/z 391.1 (M−1). ¹H NMR (400 MHz, CDCl₃) δ 1.26 (d, J=6.8 Hz, 3H), 1.30 (d, J=6.8 Hz, 3H), 3.08 (septet, J=6.8 Hz, 1H), 3.59 (br s, 1H), 3.66 (dd, J=9.4, 7.6 Hz, 1H), 3.75 (d, J=10.2 Hz, 1H), 4.13 (d, J=9.5 Hz, 1H), 4.26 (m, 1H), 4.34 (d, J=9.5 Hz, 1H), 4.41 (dd, J=9.3, 7.7 Hz, 1H), 7.32 (d, J=3.9 Hz, 1H), 7.61 (d, J=3.9 Hz, 1H), 7.66 (m, 4H). The absolute configurations of N-{(3S,4S)-4-[4-(5-cyano-2-thienyl)phenyl]-4-hydroxytetrahydrofuran-3-yl}propane-2-sulfonamide and N-{(3R,4R)-4-[4-(5-cyano-2-thienyl)phenyl]-4-hydr oxytetrahydrofuran-3-yl}propane-2-sulfonamide were tentatively assigned.

### Examples 3 and 4

### N-[(3S,4S)-4-biphenyl-4-yl-4-hydroxytetrahydrofuran-3-yl]propane-2-sulfonamide (3) and N-[(3R,4R)-4-biphenyl-4-yl-4-hydroxytetrahydrofuran-3-yl]propane-2-sulfonamide (4)

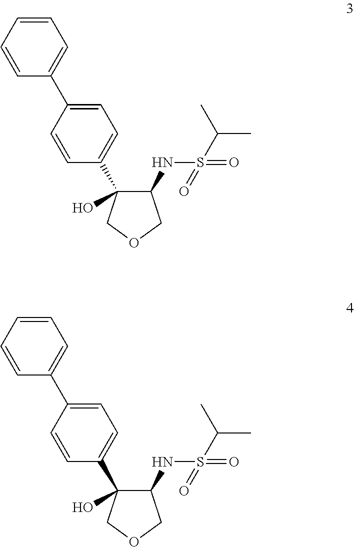

Step 1. Preparation of trans-N-(4-biphenyl-4-yl-4-hydroxytetrahydrofuran-3-yl)propane-2-sulfonamide and cis-N-(4-biphenyl-4-yl-4-hydroxytetrahydrofuran-3-yl)propane-2-sulfonamide.

A solution of N-(4-oxotetrahydrofuran-3-yl)propane-2-sulfonamide (1.08 g, 5.21 mmol) in tetrahydrofuran (10 mL) at 0° C. was treated with (biphenyl-4-yl)magnesium bromide (0.5 M solution in tetrahydrofuran, 42 mL, 21 mmol). The solution was stirred for 4 hours at 0° C., and then at room temperature for 2 days. The reaction was cooled to 0° C. and quenched with saturated aqueous ammonium chloride solution. The layers were separated, and the aqueous layer was extracted with ethyl acetate (2×10 mL). The organic layers were combined with those of an identical reaction carried out using N-(4-oxotetrahydrofuran-3-yl)propane-2-sulfonamide (105 mg, 0.507 mmol), dried over sodium sulfate, filtered, concentrated under reduced pressure and purified via silica gel chromatography (Gradient: 10% to 40% ethyl acetate in heptane) to afford a mixture of trans-N-(4-biphenyl-4-yl-4-hydroxytetrahydrofuran-3-yl)propane-2-sulfonamide and cis-N-(4-biphenyl-4-yl-4-hydroxytetrahydrofuran-3-yl)propane-2-sulfonamide. The resulting material (1.9 g) was recrystallized with 20 mL of a 1:1 mixture of diisopropyl ether and heptane, then repurified by silica gel chromatography to afford cis-N-(4-biphenyl-4-yl-4-hydroxytetrahydrofuran-3-yl)propane-2-sulfonamide as a solid. Yield: 210 mg, 0.58 mmol, 10%. LCMS m/z 360.1 (M−1). ¹H NMR (400 MHz, CDCl₃) δ 1.16 (d, J=6.8 Hz, 3H), 1.25 (d, J=6.8 Hz, 3H), 2.97 (septet, J=6.8 Hz, 1H), 3.73 (dd, J=9.3, 6.6 Hz, 1H), 3.95 (br d, J=9.5 Hz, 1H), 4.09 (d, J=9.5 Hz, 1H), 4.22 (m, 1H), 4.36 (d, J=9.5 Hz, 1H), 4.40 (dd assumed, partially obscured, J=9.3, 7.3 Hz, 1H), 7.38 (m, 1H), 7.47 (m, 2H), 7.60 (m, 4H), 7.67 (br d, J=8.5 Hz, 2H). ¹³C NMR (100 MHz, CDCl₃) δ 16.38, 54.28, 63.65, 72.17, 77.70, 81.22, 126.71, 127.04, 127.51, 127.65, 128.88, 138.01, 140.12, 141.39. The relative stereochemistry of major isomer cis-N-(4-biphenyl-4-yl-4-hydroxytetrahydrofuran-3-yl)propane-2-sulfonamide was assigned on the basis of literature work; see L. E. Overman, M. E. Okazaki and P. Mishra, Tetrahedron Letters 1986, 27, 4391-4394.

Additional fractions provided a roughly 4:1 mixture of cis-N-(4-biphenyl-4-yl-4-hydroxytetrahydrofuran-3-yl)propane-2-sulfonamide and trans-N-(4-biphenyl-4-yl-4-hydroxytetrahydrofuran-3-yl)propane-2-sulfonamide. Yield: 1.5 g, 4.15 mmol, 72%.

Step 2. Isolation of N-[(3S,4S)-4-biphenyl-4-yl-4-hydroxytetrahydrofuran-3-yl]propane-2-sulfonamide and N-[(3R,4R)-4-biphenyl-4-yl-4-hydroxytetrahydrofuran-3-yl]propane-2-sulfonamide.

Cis-N-(4-biphenyl-4-yl-4-hydroxytetrahydrofuran-3-yl)propane-2-sulfonamide isolated in the previous step (210 mg, 0.58 mmol) was separated by chiral chromatography using a Chiralpak AD column (Eluant 40:60 heptane:ethanol).

Material eluting at 7.267 minutes was collected to yield N-[(3R,4R)-4-biphenyl-4-yl-4-hydroxytetrahydrofuran-3-yl]propane-2-sulfonamide as a solid. Yield: 104 mg. LCMS m/z 360.1 (M+1). ¹H NMR (400 MHz, CDCl₃) δ 1.16 (d, J=6.8 Hz, 3H), 1.25 (d, J=6.8 Hz, 3H), 2.97 (septet, J=6.8 Hz, 1H), 3.73 (dd, J=9.3, 6.6 Hz, 1H), 3.96 (d, J=9.5 Hz, 1H), 4.09 (d, J=9.5 Hz, 1H), 4.22 (m, 1H), 4.37 (d, J=9.5 Hz, 1H), 4.40 (dd, assumed; partially obscured, J=9.3, 7.0 Hz, 1H), 7.38 (m, 1H), 7.47 (m, 2H), 7.60 (m, 4H), 7.67 (m, 2H). ¹³C NMR (100 MHz, CDCl₃) δ 16.35, 16.38, 54.28, 63.63, 72.17, 77.69, 81.22, 126.72, 127.02, 127.50, 127.66, 128.88, 138.01, 140.11, 141.38. Optical rotation: [ ]_{D}²⁵=+45.3 (c=3.4, CH₂Cl₂).

Material eluting at 11.299 minutes was collected to yield N-[(3S,4S)-4-biphenyl-4-yl-4-hydroxytetrahydrofuran-3-yl]propane-2-sulfonamide as a solid. Yield: 81.8 mg. LCMS m/z 360.1 (M+1). ¹H NMR (400 MHz, CDCl₃) δ 1.12 (d, J=6.8 Hz, 3H), 1.22 (d, J=6.8 Hz, 3H), 2.93 (septet, J=6.8 Hz, 1H), 3.42 (v br s, 1H), 3.73 (dd, J=9.3, 6.2 Hz, 1H), 4.06 (d, J=9.5 Hz, 1H), 4.11 (br d, J=9.5 Hz, 1H), 4.20 (m, 1H), 4.35 (d, J=9.5 Hz, 1H), 4.39 (dd, J=9.4, 6.9 Hz, 1H), 7.38 (m, 1H), 7.46 (dd, J=7.5, 7.5 Hz, 2H), 7.59 (m, 4H), 7.65 (d, J=8.3 Hz, 2H). ¹³C NMR (100 MHz, CDCl₃) δ 16.27, 16.32, 54.22, 63.56, 72.32, 77.49, 81.26, 126.75, 126.98, 127.40, 127.62, 128.85, 138.00, 140.08, 141.27. Optical rotation: [ ]_{D}²⁵=−53.1 (c=3.8, CH₂Cl₂).

The absolute configurations of N-[(3S,4S)-4-biphenyl-4-yl-4-hydroxytetrahydrofuran-3-yl]propane-2-sulfonamide and N-[(3R,4R)-4-biphenyl-4-yl-4-hydroxytetrahydrofuran-3-yl]propane-2-sulfonamide were tentatively assigned.

Biological Protocols

Materials and Methods

Growth and Maintenance of ES Cells

The murine ES cell line used was E14-Sx1-16C, which has a targeted mutation in the Sox1 gene, a neuroectodermal marker, that offers G418 resistance when the Sox1 gene is expressed (Stem Cell Sciences). ES cells were maintained undifferentiated as previously described (Roach). Briefly, ES cells were grown in SGML media that had a base medium of Knockout™ D-MEM (Invitrogen), supplemented with 15% ES qualified Fetal Bovine Serum (FBS) (Invitrogen), 0.2 mM L-Glutamine (Invitrogen), 0.1 mM MEM non-essential amino acids (Invitrogen), 30 μg/ml Gentamicin (Invitrogen), 1000 u/ml ESGRO (Chemicon) and 0.1 mM 2-Mercaptoethanl (Sigma). ES cells were plated on gelatin-coated dishes (BD Biosciences), the media was changed daily and the cells were dissociated with 0.05% Trypsin EDTA (Invitrogen) every other day.

Neural In Vitro Differentiation of ES Cells

Embryoid Body Formation: Prior to embryoid body (EB) formation the ES cells were weaned from FBS onto Knockout Serum Replacement (KSR) (Invitrogen). To form EBs, ES cells were dissociated into a single cell suspension, then 3×106 cells were plated in bacteriology dishes (Nunc 4014) and grown as a suspension culture in NeuroEB-I medium that consisted of Knockout™ D-MEM (Invitrogen), supplemented with 10% KSR (Invitrogen), 0.2 mM L-Glutamine (Invitrogen), 0.1 mM MEM non-essential amino acids (Invitrogen), 30 μg/ml Gentamicin (Invitrogen), 1000 u/ml ESGRO (Chemicon), 0.1 mM 2-Mercaptoethanl (Sigma) and 150 ng/ml Transferrin (Invitrogen). The plates were put on a Stovall Belly Button shaker in an atmospheric oxygen incubator. The media was changed on day 2 of EB formation with NeuroEB-I and on day 4 with NeuroEB-II (NeuroEB-I plus 1 μg/ml mNoggin [R&D Systems]).

Neuronal Precursor Selection and Expansion: On day 5 of EB formation, EBs were dissociated with 0.05% Trypsin EDTA, and 4×10⁶ cells/100 mm dish were plated on Laminin coated tissue culture dishes in Neuroll-G418 medium that consisted of a base medium of a 1:1 mixture of D-MEM/F12 supplemented with N2 supplements and NeuroBasal Medium supplemented with B27 supplement and 0.1 mM L-Glutamine (all from Invitrogen). The base medium was then supplemented with 10 ng/ml bFGF (Invitrogen), 1 μg/ml mNoggin, 500 ng/ml SHH—N, 100 ng/ml FGF-8b (R&D Systems), 1 μg/ml Laminin and 200 μg/ml G418 (Invitrogen) for selection of neuronal precursors expressing Sox-1. The plates were put in an incubator that contained 2% Oxygen and were maintained in these conditions. During the 6-day selection period, the Neuroll media was changed daily. On day 6, the surviving neuronal precursor foci were dissociated with 0.05% Trypsin EDTA and the cells were plated at a density of 1.5×10⁶ cells/100 mm Laminin coated dish in Neuroll-G418 medium. The cells were dissociated every other day for expansion, and prepared for Cryopreservation at passage 3 or 4. The crypreservation medium contained 50% KSR, 10% Dimethyl Sulfoxide (DMSO) (Sigma) and 40% Neurol-G418I medium. Neuronal precursors were cryopreserved at a concentration of 4×10⁶ cells/ml and 1 ml/cryovial in a controlled rate freezer overnight then transferred to an ultra-low freezer or liquid nitrogen for long-term storage.

Neuronal Differentiation: Cryopreserved ES cell-derived neuronal precursors were thawed by the rapid thaw method in a 37-degree water-bath. The cells were transferred from the cryovial to a 100 mm Laminin coated tissue culture dish that already contained Neuroll-G418 that had been equilibrated in a 2% Oxygen incubator. The media was changed with fresh Neuroll-G418 the next day. The cells were dissociated every other day as described above for expansion to generate enough cells to plate for the screen. For the screen, the cells were plated into 384-well poly-d-lysine coated tissue culture dishes (BD Biosciences) by the automated SelecT at a cell density of 6K cells/well in differentiation medium Neuroll that contained a 4:1 ratio of the NeuroBasalMedium/B27:D-MEM/F12/N2 supplemented with 1 μM cAMP (Sigma), 2000/1 Ascorbic Acid (Sigma), 1 μg/ml Laminin (Invitrogen) and 10 ng/ml BDNF (R&D Systems). The plates were put in an incubator with 2% Oxygen and allowed to complete the differentiation process for 7 days. The cells could then be used over a 5-day period for the high throughput screen.

In Vitro Assays

Procedure for AMPA ES Cell FLIPR Screen

FLIPR Methods and Data Analysis:

On the day of the assay, the FLIPR assay may be performed using the following methods:

Assay Buffer:

| Compound | g/L | MW | [concentration] |
|---|---|---|---|
| NaCl | 8.47 | 58.44 | 145 mM |
| Glucose | 1.8 | 180.2 | 10 mM |
| KCl | .37 | 74.56 | 5 mM |
| MgSO₄ | 1 ml 1M Stock | 246.48 | 1 mM |
| HEPES | 2.38 | 238.3 | 10 mM |
| CaCl₂ | 2 ml 1M Stock | 110.99 | 2 mM |

The pH is adjusted to 7.4 with 1M NaOH. Prepare a 2 mM (approx.) stock solution of Fluo-4, am (Invitrogen) dye in DMSO—22 μl DMSO per 50 μg vial (440 μL per 1 mg vial). Make a 1 mM (approx.) flou-4, PA working solution per vial by adding 22 μl of 20% pluronic acid (PA) (Invitrogen) in DMSO to each 50 μg vial (440 μL per 1 mg vial). Prepare a 250 mM Probenecid (Sigma) stock solution. Make 4 μM (approx.) dye incubation media by adding the contents of 2 50 μg vials per 11 ml DMEM high glucose without glutamine (220 ml DMEM per 1 mg vial). Add 110 μL probenecid stock per 11 ml media (2.5 mM final concentration). Dye concentrations ranging from 2 μM to 8 μM dye can be used without altering agonist or potentiator pharmacology. Add probenecid to the assay buffer used for cell washing (but not drug preparation) at 110 μl probenecid stock per 11 ml buffer.

Remove growth media from cell plates by flicking. Add 50 μl/well dye solution. Incubate 1 hour at 37° C. and 5% CO₂. Remove dye solution and wash 3 times with assay buffer+probenecid (100 μl probenecid stock per 10 ml buffer), leaving 30 μL/well assay buffer. Wait at least 10-15 minutes. Compound and agonist challenge additions are performed with the FLIPR (Molecular Devices). The 1^{st} addition is for test compounds, which are added as 15 μL of a 4× concentration. The second 2^{nd} addition is 15 μL of 4×concentration of agonist or challenge. This achieves 1× concentration of all compounds only after 2^{nd} addition. Compounds are pretreated at least 5 minutes before agonist addition.

Several baseline images are collected with the FLIPR before compound addition, and images are collected for least one minute after compound addition. Results are analyzed by subtracting the minimum fluorescent FLIPR value after compound or agonist addition from the peak fluorescent value of the FLIPR response after agonist addition to obtain the change in fluorescence. The change in fluorescence (RFUs, relative fluorescent units) are then analyzed using standard curve fitting algorithms. The negative control is defined by the AMPA challenge alone, and the positive control is defined by the AMPA challenge plus a maximal concentration of cyclothiazide (10 uM or 32 uM).

Compounds are delivered as DMSO stocks or as powders. Powders are solubilized in DMSO. Compounds are then added to assay drug buffer as 40 μL top [concentration] (4× the top screening concentration). The standard agonist challenge for this assay is 32 uM AMPA.

EC₅₀ values of the compounds of the invention are preferably 10 micromolar or less, more preferably 1 micromolar or less, even more preferably 100 nanomolar or less. The data for specific compounds of the invention is provided below in Table 1.

TABLE 1

| | AMPA | |
|---|---|---|
| | Potentiator | |
| Example | Assay | |
| Number | EC₅₀ | |
| 1 | 5.93 | uM* |
| 2 | >31.6 | uM |
| 3 | 1.19 | uM* |
| 4 | >31.6 | uM |

*Value represents the geometric mean of 2 EC₅₀ determinations.

When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. A compound of formula I, or a pharmaceutically acceptable salt thereof, 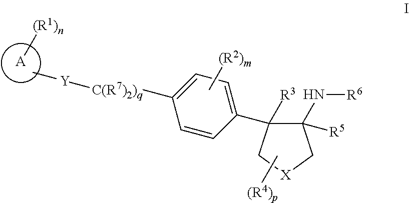
wherein each R¹ and each R² and each R⁷ is independently selected from the group consisting of hydrogen, halogen, hydroxyl, (C₁-C₆)alkoxy, cyano, nitro, amino, (C₁-C₆)alkylamino, di(C₁-C₆)alkylamino, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, (C₆-C₁₀)aryl, (C₁-C₉)heteroaryl, (C₁-C₉)heterocycloalkyl, (C₃-C₁₀)cycloalkyl, or (C₁-C₆)alkyl-S(O)₂—NH—, wherein said (C₁-C₆)alkoxy, (C₁-C₆)alkylamino, di(C₁-C₆)alkylamino, —(C═O)NH((C₁-C₆)alkyl), —(C═O)N—((C₁-C₆)alkyl)₂, —(C═O)O—(C₁-C₆)alkyl, (C₁-C₆)alkyl, (C₆-C₁₀)aryl, (C₁-C₉)heteroaryl, (C₁-C₉)heterocycloalkyl, (C₃-C₁₀)cycloalkyl or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl, (C₃-C₁₀)cycloalkyl, (C₃-C₁₀)cycloalkenyl, (C₁-C₉)heterocycloalkyl, (C₆-C₁₀)aryl, (C₁-C₉)heteroaryl, —(C═O)R⁹, —(C═O)OR⁹, —O(C═O)OR⁹, —(C═O)—N(R⁹)₂, —SO₂—N(R⁹)₂, —N(R⁹)₂, —NR⁹—(C═O)R⁹, and —N(R⁹)—S(O)₂R⁹ wherein each of the R⁸ (C₁-C₆)alkyl, (C₁-C₉)heterocycloalkyl, (C₃-C₁₀)cycloalkyl, (C₆-C₁₀)aryl or (C₁-C₉)heteroaryl is optionally independently substituted with one or more substituents independently selected from the group consisting of halogen, cyano, —R⁹, —OR⁹, —N(R⁹)₂, —S(O)ₜR⁹, —S(O)₂N(R⁹)₂, —N(R⁹)—SO₂R⁹, —O(C═O)R⁹, —(C═O)—OR⁹, —(C═O)—N(R⁹)₂, —N(R⁹)—(C═O)—R⁹, —N(R⁹)—(C═O)—N—(R⁹)₂, and —(C═O)R⁹;
t is 0, 1 or 2;
or when R¹ is (C₆-C₁₀)aryl or (C₁-C₉)heteroaryl, two R⁸ substituents bonded to adjacent carbon atoms of R¹, together with the adjacent carbon atoms, may be taken together to form a (C₁-C₉)heterocyclic or (C₃-C₁₀)carbocyclic ring which is optionally substituted with one or more R¹⁰, wherein each R¹⁹ is independently selected from the group consisting of hydrogen, —CN, halogen, —(C═O)R⁹, —(C═O)—N(R⁹)₂, —N(R⁹)₂, —OR⁹ or —R⁹;
or, two R¹ substituents bonded to adjacent carbon atoms of ring “A,” may be taken together with the adjacent carbon atoms, form a (C₁-C₉)heterocyclic or (C₃-C₁₀)carbocyclic ring which is optionally substituted with one or more R¹⁰;
m is zero, one, two or three;
n is zero, one, two or three;
p is zero, one, two or three;
q is zero, one, two or three;
R³ is hydroxyl;
each R⁴ is independently selected from the group consisting of hydrogen, hydroxyl, (C₁-C₆)alkoxy, cyano, nitro, —(C═O)NH₂, —(C═O)NH((C₁-C₆)alkyl), —(C═O)N((C₁-C₆)alkyl)₂, —O(C═O)(C₁-C₆)alkyl, —(C═O)—O—(C₁-C₆)alkyl, (C₁-C₆)alkyl, (C₁-C₆)alkyl-S(O)₂—NH— or two R⁴ groups on the same carbon atom may be taken together to form an oxo (═O) radical; wherein said (C₁-C₆)alkoxy, —(C═O)NH(alkyl), —(C═O)N-(alkyl)₂, —(C═O)O—(C₁-C₆)alkyl, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl, —(C═O)R⁹, —(C═O)OR⁹, —O(C═O)OR⁹, —(C═O)—N(R⁹)₂, —SO₂—N(R⁹)₂, —N(R⁹)₂, —NR⁹—(C═O)R⁹, and —N(R⁹)—S(O)₂R⁹ wherein each of the R⁸ (C₁-C₆)alkyl is optionally independently substituted with one or more substituents independently selected from the group consisting of halogen, cyano, —R⁹, —OR⁹, —N(R⁹)₂, —S(O)_{q}R⁹, —S(O)₂N(R⁹)₂, —N(R⁹)—SO₂R⁹, —O(C═O)R⁹, —(C═O)—OR⁹, —(C═O)—N(R⁹)₂, —N(R⁹)—(C═O)—R⁹, —N(R⁹)—(C═O)—N—(R⁹)₂, and —(C═O)R⁹;
R⁵ is hydrogen,
R⁶ is (C₁-C₆)alkyl-(C═O)—, [(C₁-C₆)alkyl]₂N—(C═O)—, (C₁-C₆)alkyl-SO₂—, (C₃-C₁₀)cycloalkyl-SO₂—, or [(C₁-C₆)alkyl]₂N—SO₂—; wherein said (C₁-C₆)alkyl moieties of said [(C₁-C₆)alkyl]₂N—(C═O)— and [(C₁-C₆)alkyl]₂N—SO₂— may optionally be taken together with the nitrogen atom to which they are attached to form a three to six membered heterocyclic ring;
R⁸ is independently selected from the group consisting of halogen, —CN, —OR⁹, (C₁-C₆)alkyl, (C₂-C₆)alkenyl, (C₃-C₁₀)cycloalkyl, (C₃-C₁₀)cycloalkenyl, (C₁-C₉)heterocycloalkyl, (C₆-C₁₀)aryl, (C₁-C₉)heteroaryl, —(C═O)R⁹, —(C═O)OR⁹, —O(C═O)OR⁹, —(C═O)N(R⁹)₂, —SO₂NR⁹, —N(R⁹)₂, —N(R⁹)—(C═O)R⁹, and —N(R⁹)₂—SO₂R⁹ wherein each of the R⁸ (C₁-C₆)alkyl, (C₁-C₉)heterocycloalkyl, (C₃-C₁₀)cycloalkyl, (C₆-C₁₀)aryl or (C₁-C₉)heteroaryl is optionally independently substituted with one or more substituents independently selected from the group consisting of halogen, cyano, —R⁹, —OR⁹, —N(R⁹)₂, —S(O)_{c},R⁹, —SO₂N(R⁹)₂, —NR⁹SO₂R⁹, —O(C═O)R⁹, —(C═O)OR⁹, —(C═O)N(R⁹)₂, —NR⁹(C═O)R⁹, —(NR⁹)—(C═O)N(R⁹)₂, and —(C═O)R⁹;
R⁹ is independently selected from the group consisting of hydrogen, (C₁-C₆)alkyl, (C₂-C₆)alkenyl, (C₂-C₆)alkynyl, (C₆-C₁₀)cycloalkyl, (C₆-C₁₀)aryl, (C₁-C₉)heterocycloalkyl and (C₁-C₆)heteroaryl; wherein each R⁹ (C₁-C₆)alkyl, (C₂-C₆)alkenyl, (C₂-C₆)alkynyl, (C₃-C₁₀)cycloalkyl, (C₆-C₁₀)aryl, (C₁-C₉)heterocycloalkyl or heteroaryl is optionally independently substituted with one or more substituents independently selected from the group consisting of halogen, hydroxy, cyano, nitro, amino, (C₁-C₆)alkylamino, di(C₁-C₆)alkylamino, (C₁-C₆)alkyl optionally substituted with one or more halogen or (C₁-C₆)alkoxy or (C₆-C₁₀)aryloxy, (C₆-C₁₀)aryl optionally substituted with one or more halogen or (C₁-C₆)alkoxy or (C₁-C₆)alkyl or trihalo(C₁-C₆)alkyl, (C₁-C₉)heterocycloalkyl optionally substituted with (C₆-C₁₀)aryl or (C₁-C₉)heteroaryl or ═O or alkyl optionally substituted with hydroxy, (C₃-C₁₀)cycloalkyl optionally substituted with hydroxy, (C₁-C₉)heteroaryl optionally substituted with one or more halogen or (C₁-C₆)alkoxy or (C₁-C₆)alkyl or trihalo(C₁-C₆)alkyl, halo(C₁-C₆)alkyl, hydroxy(C₁-C₆)alkyl, carboxy, (C₁-C₆)alkoxy, (C₆-C₁₀)aryloxy, (C₁-C₆)alkoxycarbonyl, aminocarbonyl, (C₁-C₆)alkylaminocarbonyl and di(C₁-C₆)alkylaminocarbonyl;
R¹⁰ is independently selected from the group consisting of hydrogen, —CN, halogen, —(C═O)R⁹, —(C═O)NR⁹, NR⁹, —OR⁹ or —R⁹;
ring “A” is (C₆-C₁₀)aryl, (C₁-C₉)heteroaryl, (C₄-C₁₀)cycloalkyl, or (C₁-C₉)heterocycloalkyl;
“X” is >NH, —O— or >C(R⁴)₂; and
“Y” is absent, >NR¹¹, —NR¹¹—(C═O)—, —O— or >C(R⁷)₂.

2. The compound of claim 1, or a pharmaceutically acceptable salt thereof, wherein said compound has the regiochemistry of Formula Ia:

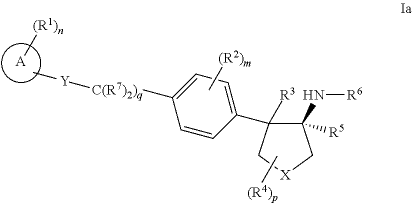

3. The compound of claim 1, or a pharmaceutically acceptable salt thereof, wherein said compound has the stereochemistry of Formula Ib:

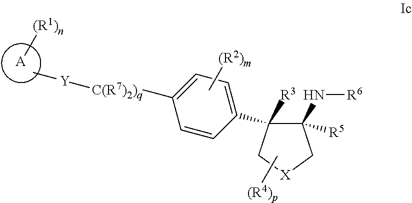

4. The compound of claim 1, or a pharmaceutically acceptable salt thereof, wherein said compound has the stereochemistry of Formula Ic:

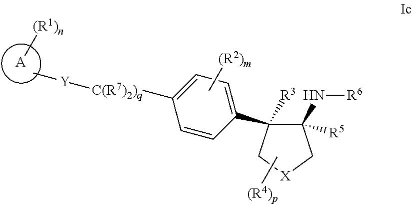

5. The compound of claim 1, or a pharmaceutically acceptable salt thereof, wherein X is —O—.

6. The compound of claim 1, or a pharmaceutically acceptable salt thereof, wherein X is >NH.

7. The compound of claim 1, or a pharmaceutically acceptable salt thereof, wherein ring A is phenyl and R¹ is in the ortho position relative to Y.

8. The compound of claim 1, or a pharmaceutically acceptable salt thereof, wherein ring A is phenyl; n is one; R¹ is in the ortho position relative to Y; and wherein R¹ is hydrogen, halogen, hydroxyl, (C₁-C₆)alkoxy, cyano, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, or (C₁-C₆)alkyl-S(O)₂—NH—, wherein said (C₁-C₆)alkoxy, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl.

9. The compound of claim 1, or a pharmaceutically acceptable salt thereof, wherein ring “A” is (C₁-C₉)heteroaryl; n is one; and wherein R¹ is hydrogen, halogen, hydroxyl, (C₁-C₆)alkoxy, cyano, —(C═O)NH₂, —(C═O)NH((C₁-C₆)alkyl), —(C═O)N((C₁-C₆)alkyl)₂, —O(C═O)—(C₁-C₆)alkyl, —(C═O)—O—(C₁-C₆)alkyl, (C₁-C₆)alkyl, or (C₁-C₆)alkyl-S(O)₂—NH—, wherein said (C₁-C₆)alkoxy, (C₁-C₆)alkyl, or (C₁-C₆)alkyl-SO₂—NH— are each independently optionally substituted with one, two, three or four R⁸, wherein each R⁸ is independently selected from the group consisting of halogen, —CN, —OR⁹, (C₁-C₆)alkyl, (C₂-C₆)alkenyl.

10. The compound of claim 1, or a pharmaceutically acceptable salt thereof, wherein R¹ is (C₁-C₆)alkoxy, (C₁-C₆)alkyl, cyano or halogen and is in the ortho or para position relative to Y.

11. The compound of claim 1, or a pharmaceutically acceptable salt thereof, wherein R² is hydrogen.

12. The compound of claim 1, or a pharmaceutically acceptable salt thereof, wherein R⁴ is hydrogen.

13. The compound of claim 1, or a pharmaceutically acceptable salt thereof, wherein p is two and both R⁴ are taken together to form oxo.

14. The compound of claim 1, or a pharmaceutically acceptable salt thereof, wherein q is zero.

15. The compound of claim 1, or a pharmaceutically acceptable salt thereof, wherein Y is absent.

16. The compound of claim 1, or a pharmaceutically acceptable salt thereof, wherein R⁶ is (C₁-C₅)alkyl-SO₂—.

17. A method for the treatment or prevention in a mammal of a condition selected from the group consisting of acute neurological and psychiatric disorders, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, attention deficit disorder, and conduct disorder, comprising administering a compound of claim 1, or a pharmaceutically acceptable salt thereof, to the mammal.

18. A pharmaceutical composition comprising a compound of claim 1, or a pharmaceutically acceptable salt thereof, and a pharmaceutically acceptable carrier.
